(19) United States
(12) Patent Application Publication
(10) Pub. No.: US 2026/0014350 A1
(43) Pub. Date: Jan. 15, 2026

(54) MEDICAL TUBE ARTICLE

(71) Applicant: ConvaTec Limited, Flintshire (GB)

(72) Inventors: Jan Uhlar, Krcava (SK); Vladimir Jenco, Michalovce (SK)

(21) Appl. No.: 19/330,247

(22) Filed: Sep. 16, 2025

Related U.S. Application Data
(63) Continuation of application No. PCT/GB2024/050720, filed on Mar. 15, 2024.
(60) Provisional application No. 63/452,864, filed on Mar. 17, 2023.

(30) Foreign Application Priority Data
Mar. 30, 2023 (GB) 2304720.2

(51) Int. Cl. A61M 25/00; A61L 2/18

(52) U.S. Cl. CPC A61M 25/0045; A61L 2/18; A61M 25/0009; A61M 25/002; A61L 2202/24; A61M 2025/0046; A61M 2205/0222; A61M 2205/0238; A61M 2205/583; A61M 2207/00; A61M 2209/06; A61M 2210/1089

(57) ABSTRACT

The invention provides a medical tube article comprising a wall extending between a distal end and a proximal end, the wall surrounding a cavity, wherein the wall comprises a first material comprising a thermoplastic or thermo-curing polymer and an amphiphilic additive; and wherein at least a portion of the wall comprises a second material comprising at least one base polymer and a colouring agent.

### TECHNICAL FIELD OF THE INVENTION

The present invention relates to medical tube articles and the method of manufacturing medical tube articles; such as catheters.

### BACKGROUND TO THE INVENTION

Medical tubing is used for a wide range of medical uses including the insertion into human orifices. A lubricious medical tube article is required in these applications and this is often achieved by coating the medical tube article or applying an additional lubricant before use. Both methods of lubricating a medical tube article can result in residual coating and/or lubricant remaining in the patient which can in turn lead to harm or irritation.

Intermittent urinary catheterisation is a process involving insertion of a urinary catheter through an individual's urethra and into their bladder, where it is retained to empty the bladder of urine for a short time period, for example only the time period that is required for emptying, after which the catheter is removed. Intermittent catheterisation is often used by patients suffering from abnormalities of the urinary system, resulting in urinary incontinence and/or a lack of control in permitting voluntary urination. Such individuals would typically make use of intermittent catheters several times a day.

Surface coatings and additives for catheters have been used to help in alleviating these issues. U.S. Pat. Nos. 10,058,638B2 and 9,186,438B2 describe the use of a catheter containing a polymer mixture of a thermoplastic or thermo-curing polymer base material and an amphiphilic block copolymer lubricious additive. The amphiphilic block copolymer contains both a hydrophobic and hydrophilic portion. The hydrophilic portion diffuses to the surface of the catheter due to incompatibility with the hydrophobic base material and provides for a lubricious surface coating. Interactions between the hydrophobic portion of the amphiphilic molecule and the base material assist in reducing migration of the amphiphilic molecule from the catheter.

Disadvantages of medical tube articles comprising amphiphilic molecules include difficulty in identifying when a catheter has been twisted and/or is being directed at the wrong angle in use, which is a common problem in patients who self-catheterise due to the angle required to do so. It would therefore be advantageous to provide an easy to insert catheter such that patients can achieve independence and freedom to self-catheterise as and when required, without having to rely on trained personnel to be present. It would also be advantageous to provide an easily self-insertable medical tube article comprising a lubricious, low-friction surface without the need for additional lubricants which can result in difficulty in application of the article, increased potential for microbial contamination, increased costs of production and lower user acceptance due to lubricant coating transferring to clothes and furnishings.

It is an aim of embodiments of the invention to overcome one or more problems of the prior art, whether expressly disclosed herein or not.

### SUMMARY OF THE INVENTION

According to a first aspect of the invention there is provided a medical tube article comprising: a wall extending between a distal end and a proximal end, the wall surrounding a cavity, wherein the wall comprises a first material comprising a thermoplastic or thermo-curing polymer and an amphiphilic additive; and wherein at least a portion of the wall comprises a second material comprising at least one base polymer and at least one colouring agent.

The medical tube article may have a substantially circular cross section. The length of the medical tube article from the proximal end to the distal end may be significantly larger than the diameter of the cross section of the medical tube article, such as, for example at least 25, 50, 100, 150, 200 or at least 250 times larger. The wall may comprise an inner surface and an outer surface wherein the inner surface is located adjacent to the cavity and the outer surface is located at the exterior of the medical tube article.

The medical tube article of the invention is advantageous because the amphiphilic additive results in a wall with a low surface coefficient of friction and the second material adds a striped guideline which does not compromise the overall lubricity of the medical tube article but does allow the user to easily determine the orientation of the medical tube article in use. The high lubricity of the medical tube article allows easy insertion of the medical tube article in use for example insertion into a human orifice, without the need for an additional lubricant. Additional lubricants for use with medical tube articles can result in unnecessary mess, additional production costs and waste therefore the medical tube article of the invention is advantageous because the presence of the amphiphilic additive avoids the requirement for an additional lubricant. The coloured second material aids in the insertion of the medical tube article by allowing the clinician or the patient to easily orient the medical tube article for easy insertion. This is particularly advantageous when a patient is self-inserting a medical tube article, for example if the patient is self-inserting an intermittent catheter. Overall, the medical tube article of the invention comprising the wall comprising the amphiphilic additive and the coloured portion of the wall is advantageous because it is both easier and safer to use by both medically and, in particular, non-medically trained individuals resulting in an easy to insert medical tube.

The first material may be transparent, substantially transparent, translucent or opaque. The second material may be translucent or opaque. A substantially transparent first material and a translucent or opaque second material may be advantageous because it may allow the user and/or the patient to observe any fluids within the cavity of the medical tube article through the transparent first material, whilst still clearly observing the coloured second material to accurately orient the medical tube article.

The thermoplastic or thermo-curing polymer may be selected from the group consisting of: polyolefin, polyvinylchloride (PVC), polyurethane (PUR), polyolefin, styrene-butadiene copolymer (SBC), styrene-ethylene-butylene-styrene copolymer (SEBS) and thermoplastic elastomers, or combinations thereof. In some embodiments the thermoplastic or thermo-curing polymer is at least partially hydrophobic. An example of a thermoplastic polymer which is both hydrophobic and hydrophilic is Estane® 52315. In some embodiments the thermoplastic or thermo-curing polymer is hydrophobic. Examples of thermoplastic polymers which are hydrophobic include Accurel™, Styroflex™, Styrolux™, Meliflex™, and Mediprene™. In some embodiments the thermoplastic or thermo-curing polymer may be Estane® 52315, Accurel™, Styroflex™, Styrolux™, MelifleX™ or Mediprene™ or any combinations thereof.

The amphiphilic additive may be an amphiphilic block copolymer of the formula A-B wherein the A-block is hydrophobic and the B-block is hydrophilic.

The A-block may be a hydrocarbon. The A-block may comprise a carbon chain of at least 5 carbon atoms, or at least 10, 15, 20, 25, 30, 35, or at least 40 carbon atoms. The A-block may be a hydrocarbon chain block of the formula CH₃CH₂(CH₂CH₂) a wherein “a” is an integer between 10 and 25. The value of “a” may be between 5-25; for instance, “a” may be 5, 6, 7, 8, 9, 10, 11, 12, 13, 14, 15, 16, 17, 18, 19, 20, 21, 22, 23, 24, or 25, or a half integer of any of the above values. The value of “a” may preferably be between 9-25; for instance, “a” may be 9, 10, 11, 12, 13, 14, 15, 16, 17, 18, 19, 20, 21, 22, 23, 24, or 25, or a half integer of any of the above values.

In some embodiments the A-block may be branched. The A-block may comprise hydrophobic hydrocarbon chains branching therefrom. The hydrophobic hydrocarbon chains may be of shorter chain lengths than the hydrophobic hydrocarbon A-block.

In embodiments wherein the thermoplastic or thermo-curing polymer is at least partially hydrophobic, the A-block may be fully compatible with the hydrophobic parts of the thermoplastic or thermo-curing polymer such that the A-block is fully mixed into the thermoplastic or thermo-curing polymer.

The B-block may comprise at least one oxygen. The B-block may comprise at least one alcohol group. In some embodiments, the B-block is a hydrophilic oligomer comprising at least 1, 2, 3, 4, or at least 5 monomer units. In some embodiments, the B-block comprises no greater than 20, 19, 18, 17, 16, 15, 14, 13, 12, 11, 10, 9, 8, 7, or no greater than 6 monomer units. In some embodiments, the B-block comprises between 2 and 15 monomer units, preferably between 2 and 10 monomer units. At least one monomer unit may be selected from the group comprising: alkylene oxides, alkylene glycols, epihalohydrins, unsaturated carboxylic acids, alkylene imines, lactones, vinyl alcohol, and vinyl alkanoates. At least one monomer unit may be preferably selected from the group comprising: ethylene oxide, propylene oxide, ethylene glycol, propylene glycol, epichlorohydrin, acrylic acid, methacrylic acid, ethylene imine, caprolactone, vinyl alcohol, and vinyl acetate. In some embodiments, at least one monomer unit comprises alkylene oxide groups independently selected from ethylene oxide and propylene oxide, and in preferred embodiments, all of the monomer units are ethylene oxide or all of the monomer units are propylene oxide. The hydrophilic B-block may comprise further hydrophilic B-blocks branching therefrom.

The first material may comprise at least 2 amphiphilic additives. The first material may comprise at least 3, 4 or at least 5 amphiphilic additives.

The amphiphilic additive may be at least one additive selected from the group consisting of: Irgasurf™, Aquazol™ and Unithox™.

In some embodiments, the amphiphilic additive is present in the first material at a concentration of at least 0.1% by weight of the combination of thermoplastic or thermo-curing polymer and amphiphilic additive. In some embodiments, the amphiphilic additive is present at a concentration of at least 0.2%, 0.3%. 0.4%. 0.5%, 0.75%, 1%, 2%, 3%, 4%, 5%, 10%, 15% or at least 20% by weight of the combination of thermoplastic or thermo-curing polymer and amphiphilic additive. The amphiphilic additive may be present at a concentration of between 0.1% and 20% by weight, and more preferably between 0.5% and 15% by weight or between 0.5% and 5% by weight of the combination of thermoplastic or thermo-curing polymer and amphiphilic additive.

In some embodiments, the amphiphilic additive is homogenously distributed within the thermoplastic or thermo-curing polymer. The amphiphilic additive may be uniformly distributed throughout the thermoplastic or thermo-curing polymer of the medical tube article wall.

In some embodiments at least some of the amphiphilic additive may be at or on the outer surface of the wall. By “at the outer surface”, it is meant that at least a portion of the amphiphilic additive forms part of the surface or protrudes from the surface. In some embodiments part of the amphiphilic additive is retained or anchored in the wall while part of the amphiphilic additive forms part of or protrudes from the outer surface of the wall. At least part of the hydrophilic portion of the amphiphilic additive may protrude from or form part of the outer surface of the wall, while at least part of the hydrophobic portion may be retained or anchored within the wall. The part of the amphiphilic additive which protrudes from or forms part of the outer surface of the wall may be advantageous because it results in a lubricious outer surface of the wall without the need for an additional lubricant.

The amphiphilic additive may be concentrated at or on the outer surface of the body. At least 25% of the number of molecules of the amphiphilic additive may be at or on the outer surface of the body. At least 30%, 35%, 40%, 45%, 50%, 55%, 60%, 65%, 70%, 75%, 80%, 85%, 90%, or at least 95% of the number of molecules of the amphiphilic additive may be at or on the outer surface of the body.

The amphiphilic additive may be concentrated at or on the outer surface of the body. At least 25% of the number of molecules of the amphiphilic additive may have hydrophilic portions which are at or on the outer surface of the body. At least 30%, 35%, 40%, 45%, 50%, 55%, 60%, 65%, 70%, 75%, 80%, 85%, 90%, or at least 95% of the number of molecules of the amphiphilic additive may have hydrophilic portions which are at or on the outer surface of the body.

In some embodiments at least 50% of the wall of the medical tube article comprises the first material comprising the thermoplastic or thermo-curing polymer and the amphiphilic additive. At least 55%, 60%, 65%, 70%, 75% or at least 80% of the wall of the medical tube article may comprise the first material. In some embodiments at least 80%, 82%, 84%, 86%, 88%, 90%, 92%, 94%, 96%, 98% or at least 99% of the wall of the medical tube article comprises the first material. In some embodiments between 50% and 99% of the wall of the medical tube article comprises the first material. In some embodiments between 60% and 99%, or between 70% and 99%, or between 80% and 99%, or between 90% and 99% of the wall of the medical tube article comprises the first material.

In some embodiments the portion of the wall comprising the second material comprising at least one base polymer and at least one colouring agent is at least 1% of the wall of the medical tube article. In some embodiments the portion of the wall comprising the second material is at least 2%, 3%, 4%, 5%, 6%, 7%, 8%, 9%, 10%, 12%, 14%, 16%, 18%, 20%, 30%, 40% or at least 50% of the wall of the medical tube article. In some embodiments the portion of the wall comprising the second material is between 1% and 50% or between 1 and 30%, or between 1 and 20%, or between 1 and 10% of the medical tube article.

In some embodiments there may be at least 2, 3, 4 or at least 5 portions of the wall comprising the second material.

The second material forms a coloured portion of the wall of the medical tube article. The coloured portion of the wall may be a coloured stripe extending along at least a portion of the tube wall. In some embodiments the second material extends along at least a portion of a longitudinal axis of the tube. In some embodiments the second material extends along at least a portion of the wall from at or near the distal end of the medical tube article to at or near the proximal end of the medical tube article. In some embodiments the second material extends from the distal end to the proximal end of the medical tube article. In some embodiments the second material extends along 25%, 50%, 75%, 80%, 85%, 90%, 95%, 96%, 97%, 98%, 99% or 100% of the length of the medical tube article. In some embodiments the second material in the form of a stripe extends along 25%, 50%, 75%, 80%, 85%, 90%, 95%, 96%, 97%, 98%, 99% or 100% of the length of the medical tube article. The stripe may be at least 1 mm, 2 mm, 3 mm, 4 mm or at least 5 mm thick. The stripe may be continuous or discontinuous. For example, the stripe may comprise a plurality of coloured areas (such as dots or dashes for example) separated by gaps comprising the second material base polymer or polymers. The stripe may extend around at least 1 mm, 2 mm, 3 mm, 4 mm or at least 5 mm circumferentially around the tubular wall.

The second material may form a continuous unbroken coloured line extending along at least a portion of the wall. In alternative embodiments the second material may form a perforated coloured line extending along at least a portion of the wall. The second material may form a dotted or dashed coloured line extending along at least a portion of the wall.

In preferred embodiments the second material is at least partially embedded within the first material. The second material may be at least partially embedded within the first material such that it extends from the inner surface or the outer surface radially into the wall of the medical tube and is at least partially surrounded by a first material. The second material may be at least partially embedded within the first material and at least one surface of the second material may be flush with the outer surface of the tube. The second material may be at least partially embedded within the first material and at least one surface of the second material may be flush with the inner surface of the tube.

The second material may be at least partially embedded within the first material such that it extends from the outer surface of the tube at least part way towards the inner surface of the tube. The second material may be at least partially embedded within the first material such that it extends from the outer surface or the inner surface and through at least 20% of the thickness of the wall. The second material may be embedded within the first material such that it extends from the outer surface or inner surface through at least 30%, 40%, 50%, 60%, 70%, 80%, or at least 90% of the thickness of the wall. The second material may be embedded within the first material such that it extends the entire way from the outer surface of the tube to the inner surface of the tube.

In preferred embodiments the second material is a stripe at least partially embedded within the first material. The second material may be a stripe at least partially embedded within the first material such that it extends from the inner or the outer surface radially into the wall of the medical tube and is at least partially surrounded by the first material. The second material may be a stripe at least partially embedded within the first material wherein at least one surface of the stripe is flush with the outer surface of the tube. The second material may be a stripe at least partially embedded within the first material wherein at least one surface of the stripe is flush with the inner surface of the wall.

The second material may be a stripe embedded within the first material such that it extends from the outer surface or the inner surface of the wall and through at least 20% of the thickness of the wall. The second material may be a stripe embedded within the first material such that it extends from the outer surface or the inner surface of the wall through at least 30%, 40%, 50%, 60%, 70%, 80%, or at least 90% of the thickness of the wall. The second material may be a stripe embedded within the first material such that it extends the entire way from the outer surface of the wall to the inner surface of the wall.

In some embodiments the wall of the medical tube article may comprise a groove. The groove may extend radially into the wall of the medical tube. The groove may extend through at least 20% of the thickness of the wall. The groove may extend through at least 30%, 40%, 50%, 60%, 70%, 80%, or at least 90% of the thickness of the wall. The groove may extend radially from the outer surface towards the inner surface of the wall. The groove may extend radially from the inner surface towards the outer surface of the wall. In some embodiments the groove may extend along a longitudinal axis of the tube. In some embodiments the groove may extend along at least a portion of a longitudinal axis of the tube. In some embodiments the groove may extend along a longitudinal axis of the tube from the proximal to the distal end of the tube. In some embodiments the groove may extend from the proximal to the distal end of the tube and may cover at least 25%, 50%, 75%, 80%, 85%, 90%, 95%, 96%, 97%, 98%, 99% or 100% of the length of the tube. In preferred embodiments the groove may be located in the first material such that the groove may extend radially into the first material. The second material may be located in the groove. The second material may be located in the groove such that the second material may be embedded within the first material. The groove may comprise an angled cross-section such as a quadrilateral cross-section. The groove may comprise a curved cross-section such as a semicircular cross-section.

The second material may extend through the entire thickness of the wall of the medical tube article such that it extends from the inner surface of the tube to the outer surface of the tube. The second material may extend through at least part of the thickness of the wall such that it extends from the outer surface of the wall towards the inner surface of the wall. The second material may extend radially from the outer surface of the wall and through at least part of the thickness of the wall. The second material may extend radially from the inner surface of the wall and through at least part of the thickness of the wall. In some embodiments the portion of the wall comprising the second material may extend through at least 50% of the thickness of the wall of the medical tube article such that it extends from the outer surface of the wall towards the inner surface of the wall. In some embodiments the portion of the wall comprising the second material may extend through at least 55%, 60%, 65%, 70%, 75%, 80%, 85%, 90%, 95%, 96%, 97%, 98% or at least 99% of the thickness of the wall of the medical tube article such that it extends from the outer surface of the wall towards the inner surface of the wall.

In preferred embodiments the second material may be a stripe extending along at least a portion of a longitudinal axis of the wall wherein the stripe extends through at least part of the thickness of the wall of the medical tube article such that it extends from the outer surface of the wall towards the inner surface of the wall.

In preferred embodiments the second material may be a stripe extending along at least a portion of a longitudinal axis of the tube wherein the stripe extends through at least 50% of the thickness of the wall such that it extends from the outer surface of the wall towards the inner surface of the wall.

In preferred embodiments the second material may be a stripe embedded within the first material wherein the stripe extends along at least a portion of a longitudinal axis of the tube and wherein the stripe extends through at least 50% of the thickness of the wall such that it extends from the outer surface of the wall towards the inner surface of the wall.

In preferred embodiments the second material may be a stripe embedded within the first material wherein at least one surface of the second material is flush with the outer surface or the inner surface of the wall and extends through at least 50% of the thickness of the wall of the medical tube article such that it extends from the outer surface towards the inner surface, or from the inner surface towards the outer surface and wherein the second material extends along at least a portion of a longitudinal axis of the wall.

In preferred embodiments the second material may be a stripe embedded within the first material wherein at least one surface of the second material is flush with the outer surface or the inner surface of the wall and extends through at least 50% of the thickness of the wall such that it extends from the outer surface towards the inner surface, or from the inner surface towards the outer surface and wherein the second material extends along at least 25%, 50%, 75%, 80%, 85%, 90%, 95%, 96%, 97%, 98%, 99% or 100% of the length of the wall.

In preferred embodiments the second material of the wall may be a stripe extending along at least a portion of a longitudinal axis of the tube wall wherein the stripe may extend through the entire thickness of the wall of the medical tube article such that it extends from the inner surface of the tube to the outer surface of the tube.

In preferred embodiments the second material of the wall may be a stripe extending along at least 25%, 50%, 75%, 80%, 85%, 90%, 95%, 96%, 97%, 98%, 99% or 100% of the length of the tube wherein the stripe may extend through the entire thickness of the wall of the medical tube article such that it extends from the inner surface of the tube to the outer surface of the tube.

The base polymer of the second material may be a thermoplastic polymer. The base polymer of the second material may be a thermo-curing polymer. The base polymer may be an elastomer. The base polymer may be selected from the group consisting of: polyolefin, polyvinylchloride (PVC), polyurethane (PUR), polyolefin, styrene-butadiene copolymer (SBC), styrene-ethylene-butylene-styrene copolymer (SEBS) and thermoplastic elastomers, or combinations thereof.

The base polymer may be a different chemistry to the thermoplastic or thermo-curing polymer of the first material. The base polymer may be the same chemistry as the thermoplastic or thermo-curing polymer of the first material. The base polymer may comprise the same thermoplastic or thermo-curing polymer and the amphiphilic additive of first material according to the embodiments described above. Embodiments of the invention wherein the second material comprises the thermoplastic or thermo-curing polymer and the amphiphilic additive and the colouring agent may be advantageous because the colouring agent may not inhibit the migration of the amphiphilic additive in the second material. This may be advantage because the amphiphilic additive may form a lubricious surface on the portion of the wall comprising the second material such that the coefficient of the first and the second material are low to prevent rubbing or discomfort when inserting the medical tube article in use.

The base polymer of the second material may be the same or similar polarity to the thermoplastic or thermo-curing polymer of the first material. A base polymer with the same or similar polarity to the thermoplastic or thermo-curing polymer may be advantageous because it would allow migration of the amphiphilic additive of the first portion of the tube, across or within the coloured portion of the wall such that the amphiphilic additive may form a lubricious outer surface on the portion of the tube comprising the second material. The migration of the amphiphilic additive across the first and second material is advantageous because it ensures that the entire surface of the medical tube article may comprise a lubricious surface with a low coefficient of friction thereby easing the insertion of the medical tube article into an orifice in use without portions of the tube catching or rubbing on the orifice resulting in pain and/or discomfort on insertion.

In some embodiments the base polymer of the second material is according to any one of the above mentioned embodiments of the first material of the medical tube article (i.e., it comprises a thermoplastic or thermo-curing polymer and an amphiphilic additive). This embodiment may be advantageous because the amphiphilic additive may migrate within the first and second material to the outer surface of the medical tube article to create a lubricious surface. The migration of the amphiphilic additive may not be impacted by the colouring agent within the second material therefore resulting in a medical tube article with a low coefficient of friction and a coloured guideline to assist with insertion of the tube.

The or each colouring agent may impart any colour including black, white and intermediate greys. The colouring agent may be any suitable colouring agent for use in medical devices, preferably class III medical devices as defined by the FDA. In some embodiments the colouring agent may impart a green colour, a red colour, a yellow colour, a violet colour, an orange colour or a black colour. In preferred embodiments the colouring agent may impart a blue colour. A blue portion of the medical tube article may be advantageous because it is not similar in colour to any bodily fluids such that it visually stands out when the medical tube may comprise bodily fluids in the cavity, in use.

The or each colouring agent may comprise an organic or an inorganic compound or a combination thereof. The or each colouring agent may comprise a polycyclic compound. The or each colouring agent may independently comprise a compound comprising at least one chemical group selected from the group consisting of: monoazo, diazo, anthraquinone, isoindolonone, benzimidazolone, diketo pyrrolo pyrrole (DPP), naphthol, quinacridone, phthalocyanine, dioxazine, quinacridone, triphenylmethane, perylene, disodium salt or combinations thereof. The or each colouring agent may comprise a metal complex compound. The metal complex compound may comprise zinc or titanium.

The or each colouring agent may comprise or be a pigment. A pigment is defined as an insoluble colour additive which forms a suspension in water. In this application the pigment may form a suspension in the base polymer such that the base polymer is coloured.

The or each colouring agent may be a dye. A dye is defined as a colour additive which is at least partially soluble in water. In this application the dye may be soluble in the base polymer such that the base polymer is coloured. In some embodiments the dye may chemically bond with the base polymer such that the base polymer is coloured.

In some embodiments the or each colouring agent comprises at least one chromophore. The chromophore may be bonded to the base polymer. The chromophore may be bonded to at least one chemical structure within the base polymer. In some embodiments the colouring agent may be dissolved in a solvent. The solvent may be an alcohol, an ether, a ketone, an aliphatic hydrocarbon, an oils, a fat or a wax, or any combination thereof.

The or each colouring agent may be independently selected from the group consisting of: FD&C blue no. 1 (also known as brilliant blue FCF or acid blue 9), FD&C blue no. 2, D&C blue no. 4, D&C blue no. 5, FD&C green no. 3, D&C green no. 5, D&C green no. 6, FD&C red no. 4, D&C red no. 6, D&C red no. 7, D&C red no. 17, D&C red no. 21, D&C red no. 22, D&C red no. 27, D&C red no. 28, D&C red no. 30, D&C red no. 31, D&C red no. 33, D&C red no. 34, D&C red no. 36, FD&C yellow no. 5, FD&C yellow no. 6, D&C yellow no. 7, D&C yellow no. 8, D&C yellow no. 10, D&C orange no. 4, D&C orange no. 5, D&C orange no. 10, D&C orange no. 11 or D&C violet no. 2.

In some embodiments the or each colouring agent may comprise a metal oxide and a pigment. In some embodiments the or each colouring agent may comprise a metal oxide and a dye. The metal oxide may be zinc oxide. The metal oxide may be titanium oxide. This embodiment may be advantageous because the presence of a metal oxide may improve the colour stability of the second material.

The second material may comprise at least 0.01% by weight of the colouring agent or total amount of colouring agents. The second material may comprise at least 0.05%, 0.1%, 0.2%, 0.4%, 0.6%, 0.8%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or at least 10% by weight of the colouring agent or total amount of colouring agents. The second material may comprise no more than 20%, 18%, 16%, 14%, 12%, 11% or no more than 10.5% by weight of the colouring agent or total amount of colouring agents.

The wall of the medical tube article may have a thickness of at least 0.1 mm. The wall of the medical tube article may have a thickness of at least 0.2 mm, 0.3 mm, 0.4 mm, 0.5 mm, 0.6 mm, 0.7 mm, 0.8 mm, 0.9 mm, 1 mm, 1.5 mm or at least 2 mm.

The cross section of the medical tube article may be substantially circular.

The medical tube article may be for insertion into human orifices, such as the trachea, the oesophagus, the rectum, the vagina or the urethra.

The medical tube article may be a catheter.

The catheter may be a feeding catheter, an intestinal catheter, a suction catheter or a urine catheter. The catheter may be an intravenous catheter or a vascular catheter. The catheter may be an intermittent catheter. The catheter may be an indwelling catheter. The catheter may be a single-use catheter.

In preferred embodiments the catheter is an intermittent urinary catheter. The catheter may be a coude-tip or curved tip catheter, or a catheter with any other non-linear tip which requires maintenance of orientation of the tip during insertion, such as, for example, olive tip catheters, Tiemann tip catheters and tapered coude tip catheters. Thus, either the proximal or distal end of the wall may be curved or comprise a tip which is curved. One of the distal or proximal ends of the wall may therefore be curved or project at an angle from the longitudinal axis of the tubular wall. The wall of the catheter may narrow in circumference towards the proximal or distal end, preferably towards the end comprising the curved-tip or coude-tip. The coude tip or curved tip itself may taper or narrow towards its end. The coloured portion of the wall or coloured stripe may extend to the end of the coude tip or curved tip, but preferably extends up to the start of the curvature of the coude tip or curved tip. In this way the coude tip or curved tip itself may not include any second material (or coloured portion/stripe).

The coloured second material may be advantageous when used in intermittent urinary catheters because the intermittent urinary catheters may be self-inserted by the patient and therefore the coloured second material may help the patient orient the catheter easily and insert it without the need of an additional lubricant. The coloured second material may be particularly advantageous for coude-tip or curved tip catheters because it would allow the user to orient the tip of the catheter to prevent discomfort or internal damage. The coloured second material and the lubricious surface resulting from the amphiphilic additive result in a catheter which is easy to orient and insert without the requirement of an additional additive. In particular the catheter is easy to self-insert which benefits patients who regularly need to self-insert catheters.

In some embodiments either the distal or the proximal end of the medical tube article is attached to a container or a pouch. In some embodiments either the distal or the proximal end of the medical tube article is attached a needle or a cannula. In some embodiments the distal and/or the proximal end of the medical tube article comprises a connector suitable for connecting to a container, a pouch or a cannula.

The surface of the wall comprising the first material may have a surface coefficient of friction of no more than 0.5. The surface of the wall comprising the first material may have a surface coefficient of friction of no more than 0.45, 0.4, 0.35, 0.3, 0.25, 0.2, 0.19 or no more than 0.18. In preferred embodiments the surface of the wall comprising the first material has a surface coefficient of friction of no more than 0.17. The surface of the wall comprising the first material may have a surface coefficient of friction of no more than 0.165, 0.16, 0.155, 0.15, 0.14, 0.13, 0.12, 0.11, 0.1, 0.09, 0.08 or 0.07.

The surface of the wall comprising the second material may have a surface coefficient of friction of no more than 0.5. The surface of the wall comprising the second material may have a surface coefficient of friction of no more than 0.45, 0.4, 0.35, 0.3, 0.25, 0.2, 0.19 or no more than 0.18. In preferred embodiments the surface of the wall comprising the second material has a surface coefficient of friction of no more than 0.17. The surface of the wall comprising the second material may have a surface coefficient of friction of no more than 0.165, 0.16, 0.155, 0.15, 0.14, 0.13, 0.12, 0.11, 0.1, 0.09, 0.08 or 0.07.

The outer surface of the medical tube article may have a surface coefficient of friction of no more than 0.5. The outer surface of the medical tube article may have a surface coefficient of friction of no more than 0.45, 0.4, 0.35, 0.3, 0.25, 0.2, 0.19 or no more than 0.18. In preferred embodiments the outer surface of the medical tube article has a surface coefficient of friction of no more than 0.17. The outer surface of the medical tube article may have a surface coefficient of friction of no more than 0.165, 0.16, 0.155, 0.15, 0.14, 0.13, 0.12, 0.11, 0.1, 0.09, 0.08 or 0.07. Low surface coefficient of friction may be advantageous because it allows easy insertion of the medical tube article without the need for an additional lubricant.

In some preferred embodiments there is provided a medical tube article comprising a wall extending between a distal end and a proximal end, the wall surrounding a cavity, wherein the wall comprises a first material comprising a thermoplastic or thermo-curing polymer and an amphiphilic additive; wherein at least a portion of the wall comprises a second material comprising at least one base polymer and at least one colouring agent; and wherein the second material is at least partially embedded in the first material.

In some preferred embodiments there is provided a medical tube article comprising a wall extending between a distal end and a proximal end, the wall surrounding a cavity, wherein the wall comprises a first material comprising a thermoplastic or thermo-curing polymer and an amphiphilic additive; wherein at least a portion of the wall comprises a second material comprising at least one base polymer and at least one colouring agent; and wherein the second material may be a stripe and wherein the stripe is at least partially embedded in the first material.

In some preferred embodiments there is provided a medical tube article comprising a wall extending between a distal end and a proximal end, the wall surrounding a cavity, wherein the wall comprises a first material comprising a thermoplastic or thermo-curing polymer and an amphiphilic additive; wherein at least a portion of the wall comprises a second material comprising at least one base polymer and at least one colouring agent; and wherein the amphiphilic additive is an amphiphilic block copolymer chemistry A-B wherein the A-block is hydrophobic and the B-block is hydrophilic.

In some preferred embodiments there is provided a medical tube article comprising a wall extending between a distal end and a proximal end, the wall surrounding a cavity, wherein the wall comprises a first material comprising a thermoplastic or thermo-curing polymer and an amphiphilic additive; wherein at least a portion of the wall comprises a second material comprising at least one base polymer and at least one colouring agent; wherein the amphiphilic additive is an amphiphilic block copolymer chemistry A-B wherein the A-block is hydrophobic and the B-block is hydrophilic and wherein the A-block is a hydrocarbon chain block of the formula CH₃CH₂(CH₂CH₂) a where “a” is between 10 and 25, and the B-block is an oligomer.

In some preferred embodiments there is provided a medical tube article comprising a wall extending between a distal end and a proximal end, the wall surrounding a cavity, wherein the wall comprises an inner surface and an outer surface wherein the inner surface is located adjacent to the cavity and the outer surface is located at the exterior of the medical tube article; wherein the wall comprises a first material comprising a thermoplastic or thermo-curing polymer and an amphiphilic additive; wherein at least a portion of the wall comprises a second material comprising at least one base polymer and at least one colouring agent; and wherein the second material may be a stripe extending along at least a portion of the tube wall and may extend through at least part of the thickness of the wall of the medical tube article such that it extends from the outer surface of the wall towards the inner surface of the wall.

In some preferred embodiments there is provided a medical tube article comprising a wall extending between a distal end and a proximal end, the wall surrounding a cavity, wherein the wall comprises an inner surface and an outer surface wherein the inner surface is located adjacent to the cavity and the outer surface is located at the exterior of the medical tube article; wherein the wall comprises a first material comprising a thermoplastic or thermo-curing polymer and an amphiphilic additive; wherein at least a portion of the wall comprises a second material comprising at least one base polymer and at least one colouring agent; and wherein the second material may be a stripe extending along at least a portion of the tube wall and may extend through at least 50% of the thickness of the wall of the medical tube article such that it extends from the outer surface of the wall towards the inner surface of the wall.

In some preferred embodiments there is provided a medical tube article comprising a wall extending between a distal end and a proximal end, the wall surrounding a cavity, wherein the wall comprises an inner surface and an outer surface wherein the inner surface is located adjacent to the cavity and the outer surface is located at the exterior of the medical tube article; wherein the wall comprises a first material comprising a thermoplastic or thermo-curing polymer and an amphiphilic additive; wherein at least a portion of the wall comprises a second material comprising at least one base polymer and at least one colouring agent; and wherein the second material may be a stripe extending along at least a portion of the tube wall and may extend through the entire thickness of the wall of the medical tube article such that it extends from the inner surface of the tube to the outer surface of the tube.

In some preferred embodiments there is provided a medical tube article comprising a wall extending between a distal end and a proximal end, the wall surrounding a cavity, wherein the wall comprises a first material comprising a thermoplastic or thermo-curing polymer and an amphiphilic additive; wherein at least a portion of the wall comprises a second material comprising at least one base polymer and at least one colouring agent; and wherein the base polymer of the second material may comprise the same chemistry as the thermoplastic or thermo-curing polymer of the first material.

In some preferred embodiments there is provided a medical tube article comprising a wall extending between a distal end and a proximal end, the wall surrounding a cavity, wherein the wall comprises a first material comprising a thermoplastic or thermo-curing polymer and an amphiphilic additive; wherein at least a portion of the wall comprises a second material comprising at least one base polymer and at least one colouring agent; and wherein the base polymer of the second material may comprise the same or similar polarity to the thermoplastic or thermo-curing polymer of the first material.

In some preferred embodiments there is provided a medical tube article comprising a wall extending between a distal end and a proximal end, the wall surrounding a cavity, wherein the wall comprises an inner surface and an outer surface wherein the inner surface is located adjacent to the cavity and the outer surface is located at the exterior of the medical tube article, wherein the wall comprises a first material comprising a thermoplastic or thermo-curing polymer and an amphiphilic additive; wherein at least a portion of the wall comprises a second material comprising at least one base polymer and at least one colouring agent; and wherein the outer surface of the medical tube article may have a surface coefficient of friction of no more than 0.5.

In some preferred embodiments there is provided a medical tube article comprising a wall extending between a distal end and a proximal end, the wall surrounding a cavity, wherein the wall comprises an inner surface and an outer surface wherein the inner surface is located adjacent to the cavity and the outer surface is located at the exterior of the medical tube article, wherein the wall comprises a first material comprising a thermoplastic or thermo-curing polymer and an amphiphilic additive; wherein at least a portion of the wall comprises a second material comprising at least one base polymer and at least one colouring agent; and wherein the outer surface of the medical tube article may have a surface coefficient of friction of no more than 0.45, 0.4, 0.35, 0.3, 0.25, 0.2, 0.19 or no more than 0.18, or preferably no more than 0.17.

According to a second aspect of the invention there is provided a packaged medical tube article comprising a medical tube article according to the first aspect of the invention, contained within a packaging.

In some embodiments, the packaging may comprise a container. The packaging may comprise a storage and/or transportation container. In some embodiments, the container is rigid. In other embodiments, the container is flexible. In some embodiments, the container comprises rigid and flexible regions.

In some embodiments the packaging container may contain the medical tube article and a medium. The medium may comprise a storage or transportation medium. The medium may be a solution. The medium may be an aqueous solution. The medium may be a medium selected from at least one of a lubricating medium, a wetting medium and a sterilising medium. The medium may be a gel.

The medium may be a catheter wetting agent. The medium may encourage hydrophilic portions of amphiphilic additives within the medical tube article to seek towards an outer surface of the wall, which further enhances the lubricating effect of the amphiphilic additive.

The medium may be in direct contact with the medical tube article. The medical tube article may be at least partially submerged in the medium. Submersion of the medical tube article in the medium has been shown to confer optimal lubricity of the medical tube article.

The medical tube article may be fully submerged in the medium. In such embodiments, the medium may comprise at least 30% of the internal volume of the packaging, or at least 40%, 50%, 60%, 70%, 80%, 90%, or at least 95% of the internal volume of the packaging.

In some embodiments, the medical tube article is not fully submerged in the medium. In such embodiments, the medium may comprise no greater than 30% of the internal volume of the packaging, or no greater than 25%, 20%, 15%, 10%, 5%, 4%, 3%, 2%, or no greater than 1% of the internal volume of the container. In such embodiments, the medium may be in direct contact with at least part of the catheter, preferably with at least one surface thereof, preferably the outer surface. In some embodiments, the medium may be able to move freely within the container.

In some embodiments, the medium is contained within a separate container, such as a bag or sachet, within the packaging and is not in direct contact with the medical tube article. The medium container may be located in the medical tube article packaging. In some embodiments, the medium is contained in a medium container located in the medical tube article packaging and the medium is not in direct contact with the medical tube article. In some embodiments, the medium container is pierceable or rupturable, in use, to release the contained medium from the medium container and into direct contact with the medical tube article in the medical tube article packaging, preferably without requiring opening of the medical tube article packaging.

In some preferred embodiments there is provided a packaged medical tube article comprising a medical tube article according to the first aspect of the invention, contained within a packaging wherein the medical tube article may be at least partially submerged in a medium.

In some preferred embodiments there is provided a packaged medical tube article comprising a medical tube article according to the first aspect of the invention, contained within a packaging wherein the medical tube article may be fully submerged in a medium.

According to a third aspect of the invention there is provided a method of manufacturing a medical tube article comprising forming a tube comprising a wall extending between a distal end and a proximal end comprising:
a first material comprising a thermoplastic or thermo-curing polymer and an amphiphilic additive;
and a second material comprising a base polymer and at least one colouring agent;
wherein the second material is formed as a coloured stripe extending along at least a portion of the tube wall.

The medical tube article may be according to the first aspect of the invention.

The first material may be extruded. In some embodiments, the method comprises extruding the first material using direct, indirect, hydrostatic, or impact extrusion. In some embodiments, the method comprises extruding the first material using cold, warm, hot, or friction co-extrusion. The extrusion of the first material may employ elevated temperature and pressure.

The thermoplastic or thermo-curing polymer and/or the amphiphilic additive, preferably both, may be provided in granulate or powder form prior to extrusion. The thermoplastic or thermo-curing polymer granulate or powder may be melted prior to extrusion. The amphiphilic additive may be melted prior to extrusion. A mixture comprising the thermoplastic or thermo-curing polymer granulates or powder and the amphiphilic additive granulates may be melted prior to extrusion.

In some embodiments the amphiphilic additive may be dissolved in the thermoplastic or thermo-curing polymer prior to extrusion. In alternative embodiments the amphiphilic additive may be dispersed within the thermoplastic or thermo-curing polymer prior to extrusion.

In some embodiments, the method comprises co-extruding the first material such that the thermoplastic or thermo-curing polymer and the amphiphilic additive are extruded simultaneously.

The second material may be extruded. In some embodiments, the method comprises co-extruding the second material such that the base polymer and the or each colouring agent are extruded simultaneously. In some embodiments, the method comprises extruding the second material using direct, indirect, hydrostatic, or impact extrusion. In some embodiments, the method comprises extruding the second material using cold, warm, hot, or friction extrusion. The extrusion of the second material may employ elevated temperature and pressure.

The base polymer of the second material may be provided in granulate or powder form prior to extrusion. The base polymer granulate or powder may be melted prior to extrusion. The or each colouring agent may be provided in granulate or powder form prior to extrusion. The colouring agent may be melted prior to extrusion. A mixture comprising the base polymer granulates or powder and the colouring agent granulates may be melted prior to extrusion.

In alternative embodiments the or each colouring agent may be provided in liquid form prior to extrusion. In some embodiments the or each colouring agent may be dissolved or dispersed in a solvent such that it is provided in liquid form prior to extrusion. In some embodiments the colouring agent may be dissolved in the base polymer prior to extrusion. In alternative embodiments the colouring agent may be dispersed within the base polymer prior to extrusion.

In some embodiments, the method comprises co-extruding the second material such that the base polymer and the colouring agent are extruded simultaneously.

In some embodiments the method comprises forming the tube by co-extruding the first and second material such that the first and second material are extruded at substantially the same time. The method may comprise co-extruding a first mixture wherein the first mixture comprises the thermoplastic or thermo-curing polymer and the amphiphilic additive, and a second mixture wherein the second mixture comprises the base polymer and the colouring agent.

In some embodiments the method comprises forming the wall by extruding the first material followed by extruding the second material. In some embodiments the method comprises extruding the second material followed by extruding the first material.

In some embodiments the method comprises forming the wall by co-extruding the thermoplastic or thermo-curing polymer and the amphiphilic additive of the first material followed by co-extruding the base polymer and the colouring agent of the second material. In some embodiments the method comprises forming a wall by co-extruding the base polymer and the or each colouring agent of the second material followed by co-extruding the thermoplastic or thermo-curing polymer and the amphiphilic additive of the first material.

The method may comprise forming the tube by moulding the first material. The method may comprise forming the tube by moulding the second material. The method may comprise forming the tube by moulding the first material and moulding the second material. The moulding may be compression moulding, melt moulding, transfer moulding or injection moulding.

In some embodiments the method may comprise forming the tube by moulding the first material and extruding the second material. In some embodiments the method may comprise forming the tube by extruding the first material and moulding the second material.

The method may comprise forming a medical tube article of the first aspect of the invention, preferably a catheter.

### DETAILED DESCRIPTION OF THE INVENTION

In order that the invention may be more clearly understood embodiments thereof will now be described, by way of example only, with reference to the accompanying drawings, of which:

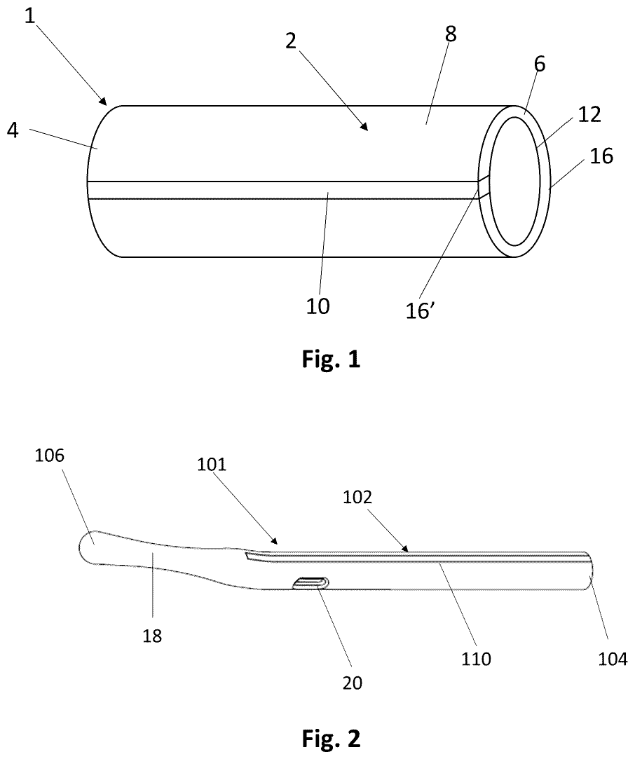

FIG. 1 illustrates a side view of a first embodiment of the medical tube article of the first aspect of the invention wherein the medical tube article comprises a first material and a second material and wherein the second material is a stripe along the medical tube article.

FIG. 2 illustrates a side view of a second embodiment of the medical tube article of the first aspect of the invention wherein the medical tube article is in the form of a Coude-tip catheter.

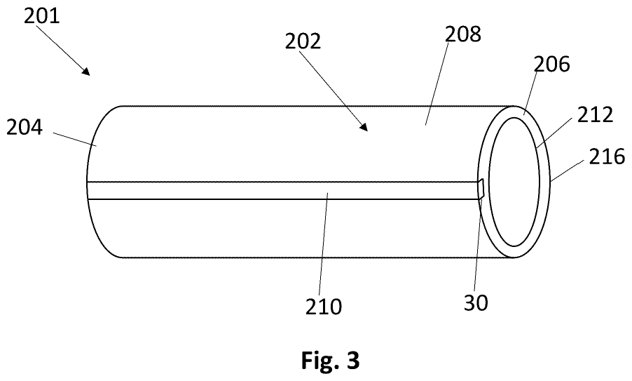

FIG. 3 illustrates a side view of a first embodiment of the medical tube article of the first aspect of the invention wherein the medical tube article comprises a first material and a second material and wherein the second material is a stripe along the medical tube article wherein the stripe extends from the outer surface through part of the thickness of the medical tube article wall.

### EXAMPLE 1

A first embodiment of a medical tube article of the first aspect of the invention is illustrated by FIG. 1. The medical tube article is in the form of a catheter (1) and comprises a wall (2) extending between a distal end (4) and a proximal end (6) wherein the wall (2) comprises a first material (8) comprising a thermoplastic polymer and an amphiphilic additive, and a portion of the wall in the form of a stripe (10) comprises a second material comprising a base polymer and a colouring agent.

The thermoplastic polymer is thermoplastic polypropylene. The amphiphilic additive is of the formula CH₃CH₂(CH₂CH₂)₁₅(OCH₂CH₂)₅OH. The first (8) material is substantially transparent. The base polymer of the second material is the same as the first material, namely the base polymer comprises thermoplastic polypropylene and the amphiphilic additive CH₃CH₂(CH₂CH₂)₁₅(OCH₂CH₂)₅OH. The colouring agent is a blue pigment. The second material and stripe (10) are substantially opaque and blue coloured.

The wall (2) of the catheter (1) comprises an inner surface (12) located at a cavity (14) in the centre of the tube and an outer surface (16) located at the exterior of the catheter (1). The stripe (10) forms a continuous coloured stripe extending from the distal end (4) to the proximal end (6). The stripe (10) extends from the inner surface (12) to the outer surface (16).

The amphiphilic additive comprises a hydrophilic block of the formula (OCH₂CH₂)₅OH which, after the medical tube article is manufactured, seeks towards the outer surface (16) due to its incompatibility with the thermoplastic polymer of the first material, resulting in the outer surface (16) becoming lubricious. The lipophilic and hydrophobic block of the amphiphilic additive of the formula CH₃CH₂(CH₂CH₂) 15 ensures that the hydrophilic block is secured to the thermoplastic polymer.

The first embodiment of the catheter (1) was manufactured according to the method of 5 the third aspect of the invention wherein the first material and the second material were co-extruded such that the second material is formed as a coloured stripe extending along at least a portion of the tube wall.

The surface coefficient of friction of the catheter (1) was measured using a Harland FTS6000 Friction Testing System (Harland Medical Systems, Inc. USA) by drawing a test sample between two silicone rubber pads clamped at a programmable force, recording the pull resistance and calculating the co-efficient of friction from the results. The catheter (1) was tested 32 times. The friction was measured immediately after exposure to X-ray and then again after 8 weeks and after 17 weeks. The data is presented in table 1.

TABLE 1

| | | Coefficient of | Coefficient of |
|---|---|---|---|
| | Coefficient of | friction after | friction after |
| | friction at 0 weeks | 8 weeks | 17 weeks |
| Test 1 | 0.0418 | 0.0806 | 0.0364 |
| Test 2 | 0.0672 | 0.1328 | 0.0336 |
| Test 3 | 0.1564 | 0.0436 | 0.0366 |
| Test 4 | 0.0446 | 0.124 | 0.0284 |
| Test 5 | 0.0702 | 0.0496 | 0.0324 |
| Test 6 | 0.0316 | 0.1128 | 0.0252 |
| Test 7 | 0.0818 | 0.0566 | 0.0314 |
| Test 8 | 0.0596 | 0.1572 | 0.0346 |
| Test 9 | 0.0856 | 0.0612 | 0.0288 |
| Test 10 | 0.0356 | 0.1272 | 0.0345 |
| Test 11 | 0.0374 | 0.0844 | 0.0306 |
| Test 12 | 0.0338 | 0.0936 | 0.0302 |
| Test 13 | 0.0344 | 0.1868 | 0.0276 |
| Test 14 | 0.0394 | 0.0942 | 0.0288 |
| Test 15 | 0.039 | 0.076 | 0.029 |
| Test 16 | 0.0342 | 0.0382 | 0.0316 |
| Test 17 | 0.0334 | 0.0458 | 0.0318 |
| Test 18 | 0.0344 | 0.036 | 0.032 |
| Test 19 | 0.0464 | 0.0442 | 0.027 |
| Test 20 | 0.035 | 0.037 | 0.1096 |
| Test 21 | 0.0338 | 0.0396 | 0.0316 |
| Test 22 | 0.0332 | 0.0386 | 0.05 |
| Test 23 | 0.0298 | 0.031 | 0.0256 |
| Test 24 | 0.0361 | 0.0316 | 0.0326 |
| Test 25 | 0.0368 | 0.0328 | 0.0266 |
| Test 26 | 0.0376 | 0.0344 | 0.0282 |
| Test 27 | 0.0332 | 0.0638 | 0.0302 |
| Test 28 | 0.0354 | 0.0324 | 0.0278 |
| Test 29 | 0.0338 | 0.033 | 0.0308 |
| Test 30 | 0.0364 | 0.0368 | 0.031 |
| Test 31 | 0.0324 | 0.0334 | 0.0284 |
| Test 32 | 0.0334 | 0.0414 | 0.0262 |

The surface coefficient of friction of the catheter (1) was not negatively impacted by the presence of the second material (10) which comprised the colouring agent. The surface coefficient of friction also remained low during the 17 week test period thereby showing that the coefficient of friction did not substantially degrade over time.

Without being bound by theory it is believed that the amphiphilic additive migrates within the first material such that it become concentrated at the outer surface of the medical tube article (16) thereby resulting in a low surface friction. It is also believed that the amphiphilic additive migrates within the base polymer of the second material and the hydrophilic block seeks towards the outer surface of the portion of the wall comprising the second material (16′). The amphiphilic additive therefore forms a lubricious layer across the entire outer surface of the wall (16), including the outer surface of the portion of the wall comprising the second material (16′).

The stripe (10) on the catheter (1) assists with the insertion of the catheter (1). The migration of the amphiphilic additive to the outer surface of the wall (16) to form a lubricious layer that covers both the first material and the stripe (10) ensures that in use the catheter (1) is lubricated without the need for an additional lubricant or an additional coating to be applied to the wall (2) and this lubricity is not negatively impacted by the additional presence of a colouring agent in the stripe (10). The enhanced lubricity of the catheter (1) as a result of the amphiphilic additive migration in combination with the coloured stripe (10), results in a catheter (1) with an improved ease of insertion into a patient.

### EXAMPLE 2

A second embodiment of a medical tube article of the first aspect of the invention is illustrated by FIG. 2. The medical tube article is in the form of a Coude-tip catheter (101) comprising a wall (102) extending between a distal end (104) and a proximal end (106) wherein the wall (102) comprises a first material comprising a thermoplastic polymer and an amphiphilic additive, and a portion of the wall in the form of a stripe (110) comprising a second material comprising a base polymer and a colouring agent (the base polymer being the same as the first material).

The coude-tip catheter (101) is identical to the first embodiment of the invention with the exception being that the proximal end of the wall (106) comprises a curved tip (18) and an aperture (20) located near the curved tip (18) and the stripe 110 does not reach the proximal end (106) of the curved tip (18) but instead ends where the curved tip starts. In embodiments not illustrated the coude-tip catheter may comprise multiple apertures located near the proximal end of the coude-tip catheter.

The coude-tip catheter (101) is manufactured by the same method as the first embodiment of the invention.

The coude-tip catheter (101) comprising the curved tip (18), the coloured stripe (110) and the amphiphilic additive is advantageous because the coloured stripe (110) allows the user to correctly orient the coude-tip catheter (101) such that it can be used to navigate the curved tip (18) around obstructions or blockages in the urethra and the amphiphilic additive ensures the coude-tip catheter (101) comprises a low surface coefficient of friction such that additional lubricant is not required. Both of these features result in a coude-tip catheter (101) with improved ease of use, especially for self-insertion which can be significantly beneficial to patients, in particular patients who may have to frequently self-insert catheters. The colouring agent in the coloured stripe (110) does not inhibit the lubricious properties of the amphiphilic additive and instead only adds further to the ease of insertion of the catheter.

### EXAMPLE 3

A third embodiment of a medical tube article of the first aspect of the invention is illustrated by FIG. 3. The medical tube article is in the form of a catheter (201) and comprises a wall (202) extending between a distal end (204) and a proximal end (206) wherein the wall (202) comprises a first material (208) comprising a thermoplastic polymer and an amphiphilic additive, and a portion of the wall in the form of a stripe (210) comprises a second material comprising a base polymer and a colouring agent wherein the stripe (210) is embedded in the first material (208).

The wall (202) comprises a groove (30) wherein the groove (30) extends radially into the wall (202) from the outer surface (216). The stripe (210) is located within the groove (30).

The third embodiment of the medical tube is substantially the same as the first embodiment with the exception that the stripe in the third embodiment does not extend the entire way from the outer surface (216) to the inner surface (212) of the wall (202) and instead extends only part of the way. This may be advantageous because it provides a catheter comprising a low coefficient of friction and a visible stripe to aid insertion of the catheter into the patient.

The above embodiments are described by way of example only. Many variations are possible without departing from the scope of the invention as defined in the appended claims.

1. A medical tube article comprising a wall extending between a distal end and a proximal end, the wall surrounding a cavity, wherein the wall comprises a first material comprising a thermoplastic or thermo-curing polymer and an amphiphilic additive; and wherein at least a portion of the wall comprises a second material comprising at least one base polymer and a colouring agent.

2. A medical tube article according to claim 1 wherein the second material is at least partially embedded in the first material.

3. A medical tube article according to claim 1 or claim 2 wherein the second material is a stripe at least partially embedded in the first material.

4. A medical tube article according to any preceding claim wherein the amphiphilic additive is an amphiphilic block copolymer chemistry A-B wherein the A-block is hydrophobic and the B-block is hydrophilic

5. A medical tube article according to claim 4 wherein the A-block is a hydrocarbon chain block of the formula CH₃CH₂(CH₂CH₂) a where “a” is between 10 and 25, and the B-block is an oligomer.

6. A medical tube article according to any preceding claim wherein the second material extends from the distal end to the proximal end of the tube.

7. A medical tube article according to any preceding claim wherein the wall comprises an inner surface and an outer surface wherein the inner surface is in contact with the cavity and wherein the second material extends through from the inner surface to the outer surface of the wall, extending through at least 50% of the thickness of the wall.

8. A medical tube article according to any preceding claim wherein between 1 and 10% by weight of the wall comprises the second material.

9. A medical tube article according to any preceding claim wherein the colouring agent is a pigment, preferably a blue pigment.

10. A medical tube article according to any one of claims 1 to 8 wherein the colouring agent is a dye, preferably a blue dye.

11. A medical tube article according to any preceding claim wherein the medical tube article has a surface friction of no more than 0.17.

12. A medical tube article according to any preceding claim wherein the thermoplastic or thermo-curing polymer and the base polymer comprise the same or similar polarity.

13. A medical tube article according to any preceding claim wherein the second material comprises an amphiphilic additive.

14. A medical tube article according to any preceding claim wherein the medical tube article is a catheter, preferably an intermittent catheter.

15. A medical tube article according to any one of claims 1 to 13 wherein the medical tube article is a feeding catheter, an intestinal catheter, a suction catheter, a urine catheter, an intravenous catheter/cannula.

16. A packaged medical tube article comprising a medical tube article according to any one of claims 1 to 15 contained within a packaging.

17. A packaged medical tube article according to claim 16 wherein at least a portion of the medical tube is in contact with a sterilising medium.

18. A packaged medical tube article according to claim 16 wherein the medical tube is at least partially submerged in the sterilising medium.

19. A packaged medical tube article according to claim 16 wherein the sterilising medium is contained in a separate container, such as a bag or sachet, located in the container and is not in direct contact with the catheter.

20. A method of manufacturing a medical tube article comprising forming a tube comprising a wall extending between a distal end and a proximal end comprising:
a first material comprising a thermoplastic or thermo-curing polymer and an amphiphilic additive;
and a second material comprising a base polymer and at least one colouring agent;
wherein the second material is formed as a coloured stripe extending along at least a portion of the tube wall.

21. A method according to claim 20 wherein the medical tube article is of any one of claims 1 to 15.

22. A method according to claim 20 or claim 21 wherein the first material is extruded and subsequently the second material is extruded.

23. A method according to claim 20 or claim 21 wherein the first material and the second material are coextruded.

24. A method according to claim 20 or claim 21 wherein the first mixture and the second material are moulded.

25. A method according to claim 20 or claim 21 wherein one of the first material and the second material is extruded and the other mixture is moulded.
